(19) United States
(12) Patent Application Publication
(10) Pub. No.: US 2024/0400516 A1
(43) Pub. Date: Dec. 5, 2024

(54) PROCESSES FOR THE PREPARATION OF 1,2,3,5,6,7-HEXAHYDRO-S-INDACENE DERIVATAIVES

(71) Applicant: Hoffmann-La Roche Inc., Little Falls, NJ (US)

(72) Inventors: Joséphine Eliette Françoise CINQUALBRE, Saint-Louis (FR); Stefan HILDBRAND, Gelterkinden (CH); Dainis KALDRE, Loerrach (DE)

(73) Assignee: Hoffmann-La Roche Inc., Little Falls, NJ (US)

(21) Appl. No.: 18/802,257

(22) Filed: Aug. 13, 2024

Related U.S. Application Data
(63) Continuation of application No. PCT/EP2023/053410, filed on Feb. 13, 2023.

(30) Foreign Application Priority Data
Feb. 15, 2022 (EP) 22156688.8

(51) Int. Cl. C07D 211/54; C07C 209/32; C07C 263/10

(52) U.S. Cl. CPC C07D 211/54; C07C 209/32; C07C 263/10

(57) ABSTRACT

The present invention relates to intermediates and processes useful for preparing 1-ethyl-N-((1,2,3,5,6,7-hexahydro-s-indacen-4-yl)carbamoyl)piperidine-4-sulfonamide and salts thereof. The present invention further relates to 1-ethyl-N-((1,2,3,5,6,7-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP3 inhibition.

### FIELD OF THE INVENTION

The present invention relates to intermediates and processes useful for preparing 1-ethyl-N-((1,2,3,5,6,7-hexahydro-s-indacen-4-yl)carbamoyl)piperidine-4-sulfonamide and salts thereof. The present invention further relates to 1-ethyl-N-((1,2,3,5,6,7-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP3 inhibition.

### BACKGROUND

1-Ethyl-N-((1,2,3,5,6,7-hexahydro-s-indacen-4-yl)carbamoyl)piperidine-4-sulfonamide is disclosed in WO 2019/008025 A1 as an NLRP3 inhibitor (see Example 6). However, there is a need to provide improved processes for preparing 1-ethyl-N-((1,2,3,5,6,7-hexahydro-s-indacen-4-yl)carbamoyl)piperidine-4-sulfonamide and salts thereof. In particular, there is a need to provide efficient processes that are suitable for large scale synthesis and which, for example, avoid multiple, complex and partially low yielding chemical steps and overall atom inefficient synthesis.

There is also a need to provide 1-ethyl-N-((1,2,3,5,6,7-hexahydro-s-indacen-4-yl)carbamoyl)piperidine-4-sulfonamide and salts thereof at a higher yield compared to prior art processes, especially on a large scale. Further, for large-scale industrialization, greener synthesis routes, reduced solvent waste, and improved safety are also of concern. The present invention solves the aforementioned problems. Furthermore, the present invention can be implemented in both batch or continuous methods.

### SUMMARY OF THE INVENTION

The invention provides a process of preparing compound (C) or a salt thereof, comprising the step of contacting compound (A) with compound (B) in the presence of a solvent and a base, to obtain compound (C) or a salt thereof.

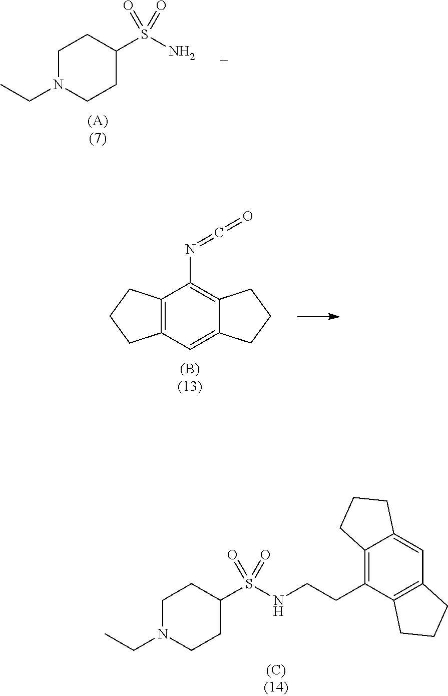 [SMILES: CCN1CCC(S(N)(=O)=O)CC1 ; O=C=Nc1c2c(cc3c1CCC3)CCC2 ; CCN1CCC(S(=O)(=O)NCCc2c3c(cc4c2CCC4)CCC3)CC1]

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

In an embodiment of the present invention, the solvent for contacting compound (A) with compound (B) is selected from toluene, anisole, cyclopentylmethylether, ethylbenzene, isopropyl acetate, isobutyl acetate, 2-methyl tetrahydrofuran, water, t-butanol, ethyl acetate, methyl acetate, xylene, tetrahydrofuran dimethyl sulfoxide, acetonitrile, t-butyl methyl ether, N-methyl pyrrolidine, N-ethyl pyrrolidone, heptane, cyclohexane, acetone, or any combination thereof.

In a further embodiment of the present invention, the solvent for contacting compound (A) with a compound (B) is selected from toluene, anisole, ethylbenzene and xylene.

In a further embodiment of the present invention, the solvent for contacting compound (A) with a compound (B) is selected from 2-methyl thetrahydrofuran and tehrahydrofuran.

In a further embodiment of the present invention, the solvent for contacting compound (A) with a compound (B) is dimethyl sulfoxide.

In a further embodiment of the present invention, the solvent for contacting compound (A) with compound (B) is toluene or toluene in combination with water, t-butanol, tetrahydrofuran, dimethyl sulfoxide or acetonitrile.

In a further embodiment of the present invention, the solvent for contacting compound (A) with compound (B) is toluene and tetrahydrofuran.

In a further embodiment of the present invention, the base for contacting compound (A) with compound (B) is selected from potassium tert-butoxide, potassium hydroxide or any other basic potassium salt.

In a further embodiment of the present invention, the base for contacting compound (A) with compound (B) is selected from potassium tert-butoxide or potassium hydroxide.

In a further embodiment of the present invention, the base for contacting compound (A) with compound (B) is potassium tert-butoxide.

An embodiment of the present invention provides a process of preparing a salt of compound (C), such as a cationic salt. Typically the salt is pharmaceutically acceptable.

For the purposes of this invention, a “cationic salt” of 1-ethyl-N-((1,2,3,5,6,7-hexahydro-s-indacen-4-yl)carbamoyl)piperidine-4-sulfonamide is a salt formed between a protic acid functionality (such as a urea proton) of the compound by the loss of a proton and a suitable cation. Suitable cations include, but are not limited to lithium, sodium, potassium, magnesium, calcium and ammonium. The salt may be a mono-, or di-salt. Preferably the salt is a mono- or di-lithium, sodium, potassium, magnesium, calcium or ammonium salt. More preferably the salt is a mono- or di-sodium salt or a mono- or di-potassium salt. More preferably the salt is a mono- or di-potassium salt, more preferably still the salt is a mono-potassium salt.

Advantageously, where a cationic salt of 1-ethyl-N-((1,2,3,5,6,7-hexahydro-s-indacen-4-yl)carbamoyl)piperidine-4-sulfonamide (Compound (C)) is desired, the cation of the salt is provided by the conjugate acid of the base. For example, one embodiment of the first aspect of the invention provides a process of preparing an alkali metal or an alkali earth metal salt of 1-ethyl-N-((1,2,3,5,6,7-hexahydro-s-indacen-4-yl)carbamoyl)piperidine-4-sulfonamide (C), comprising the step of contacting 1-ethyl-4-piperidinesulfonamide (A) with a 1,2,3,5,6,7-hexahydro-s-indacene derivative (B) or (B′) in the presence of a solvent and an alkali metal or an alkali earth metal alkoxide, to obtain the alkali metal or alkali earth metal salt of 1-ethyl-N-((1,2,3,5,6,7-hexahydro-s-indacen-4-yl)-carbamoyl)-piperidine-4-sulfonamide, wherein the alkali metal or alkali earth metal of the salt is the same as the alkali metal or alkali earth metal of the alkoxide. Typically in such an embodiment, the alkali metal or alkali earth metal alkoxide is an alkali metal or an alkali earth metal tertiary butoxide.

In an embodiment of the present invention, the salt of 1-ethyl-N-((1,2,3,5,6,7-hexahydro-s-indacen-4-yl)carbamoyl)piperidine-4-sulfonamide (C) is purified by recrystallisation or reprecipitation. For example, the crude salt of 1-ethyl-N-((1,2,3,5,6,7-hexahydro-s-indacen-4-yl)carbamoyl)piperidine-4-sulfonamide (C) may be dissolved in a first solvent to obtain a first mixture, optionally the mixture may be filtered, and the salt of the 1-ethyl-N-((1,2,3,5,6,7-hexahydro-s-indacen-4-yl)-carbamoyl)piperidine-4-sulfonamide (C) may be precipitated by the addition of a second solvent, optionally with cooling. Typically, the first solvent is a polar protic solvent such as methanol. Typically, the second solvent is a polar aprotic solvent such as acetonitrile.

A further aspect of the invention provides a process of preparing compound (C) or a salt thereof, comprising the step of contacting compound (A) with compound (B) in the presence of a solvent and a base, to obtain compound (C) or a salt thereof, wherein compound (B) is obtained from compound (D):

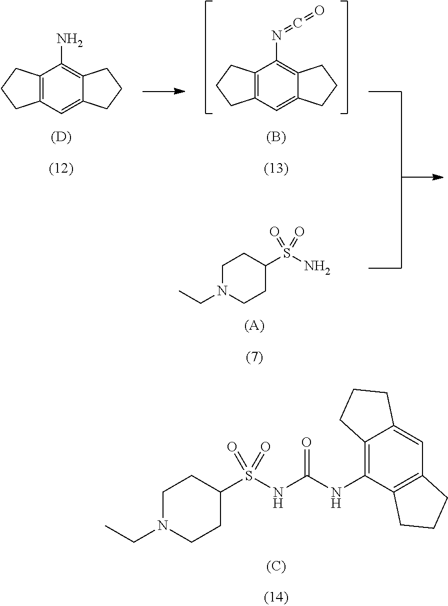

In an embodiment of the present invention, compound (C) is isolated using an antisolvent.

In a further aspect of the present invention, compound (C) is isolated using an antisolvent, wherein the antisolvent is selected from acetonitrile, any alcohol or water.

In an embodiment of the present invention, compound (C) is isolated using a wash solvent.

In a further aspect of the present invention, compound (C) is isolated using a wash solvent, wherein the wash solvent is selected from tetrahydrofuran, toluene, dimethylsulfoxide, or acetonitrile.

A second aspect of the invention provides 1-ethyl-N-((1,2,3,5,6,7-hexahydro-s-indacen-4-yl)carbamoyl)piperidine-4-sulfonamide (Compound (C)) or a salt thereof, prepared by a process of the first aspect of this invention.

In one embodiment, the second aspect of the invention provides an alkali metal or an alkali earth metal salt of 1-ethyl-N-((1,2,3,5,6,7-hexahydro-s-indacen-4-yl)carbamoyl)-piperidine-4-sulfonamide. Typically, the second aspect of the invention provides a potassium salt of 1-ethyl-N-((1,2,3,5,6,7-hexahydro-s-indacen-4-yl)carbamoyl)-piperidine-4-sulfonamide. Most typically, the second aspect of the invention provides a mono-potassium salt of 1-ethyl-N-((1,2,3,5,6,7-hexahydro-s-indacen-4-yl)carbamoyl)-piperidine-4-sulfonamide.

In an embodiment of the present invention, compound (B) is prepared by a process according to the third aspect of the invention.

A third aspect of the invention provides a process of preparing a compound (B), the process comprising the step wherein compound (D) is converted into compound (B):

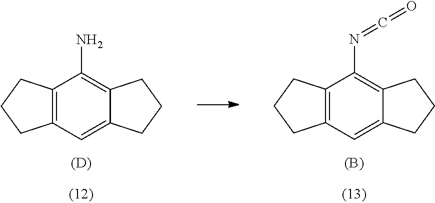

Accordingly, in one embodiment of the third aspect of the invention, there is provided a process of preparing compound (B), the process comprising the step of converting compound (D) into compound (B) using a reaction mixture of compound (D) with phosgene, triphosgene, carbonyldiimidazole, or di-tert-butyl dicarbonate in the presence of a base and a solvent.

In a further embodiment of the third aspect of the invention, the solvent is selected is selected from toluene, anisole, cyclopentylmethylether, ethylbenzene, isopropyl acetate, isobutyl acetate, 2-methyl tetrahydrofuran, water, ethyl acetate, methyl acetate, xylene, tetrahydrofuran or dimethyl sulfoxide, acetonitrile, t-butyl methyl ether, diethyl ether, dichloromethane, 1,2-dichloroethane, chloroform, N-methyl pyrrolidine, N-ethyl pyrrolidone, heptane, cyclohexane or any combination thereof, and the base is a tertiary amine, such as N,N-diisopropylethylamine, triethylamine, or tributylamine or the base is an inorganic base such as potassium carbonate, potassium hydroxide, or sodium carbonate.

In a further embodiment of the third aspect of the invention, the solvent is selected from toluene or toluene in combination with water, acetonitrile, or tetrahydrofuran, and the base is selected from N,N-diisopropylethylamine, trimethylamine, tributylamine, potassium carbonate, potassium hydroxide, or sodium carbonate.

In a further embodiment of the third aspect of the invention, the solvent is toluene and/or water, and the base is N,N-diisopropylethylamine, trimethylamine or potassium carbonate.

In a further embodiment of the third aspect of the invention, the solvent is toluene and the base is N,N-diisopropylethylamine or potassium carbonate.

In a further embodiment of the third aspect of the invention, the solvent is toluene and the base is potassium carbonate.

In a further embodiment of the third aspect of the invention, the solvent is toluene and the base is N,N-diisopropylethylamine.

Toluene and potassium carbonate, or toluene and N,N-diisopropylethylamine, provide an advantage over using THF (tetrahydrofuran) and TEA (triethylamine), as was used previously (EGGLER J F ET AL: Journal of Labelled Compounds and Radiopharmaceuticals, vol. 45, no. 9, 2002, pages 785-794, XP002264662), due to the proposed process above involving less time and energy for workup because of the elimination of the need to evaporate or perform a silica gel filtration. Therefore the process here is a less intensive process than that previously reported.

In another embodiment of the third aspect of the invention, there is provided a process of preparing compound (B), the process comprising the step of converting compound (D) into compound (B), wherein the reaction mixture is washed using an aqueous solution to obtain compound (B) in an organic solvent.

An embodiment of the present invention provides a process to obtain compound (C) wherein compound (B) is obtained according to the third aspect of the invention.

An embodiment of the present invention provides a process to obtain compound (C) according to the first aspect of the invention, wherein compound (B) is obtained according to the third aspect of the invention.

In an embodiment of the present invention, compound (B) is prepared by a batch process or in a continuous mode.

In an embodiment of the present invention, compound (B) is prepared in a continuous mode.

In an embodiment of the present invention, the process to obtain compound (B) and compound (C) are telescoped.

A fourth aspect of the invention provides compound (B):

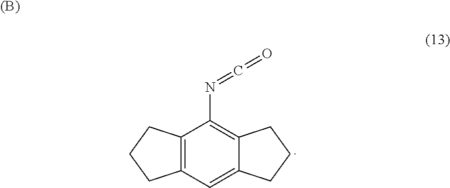 [SMILES: "(B)" ; O=C=Nc1c2c(cc3c1CCC3)CCC2]

In one embodiment of the third aspect of the invention, compound (D) is prepared by a process comprising the following steps:

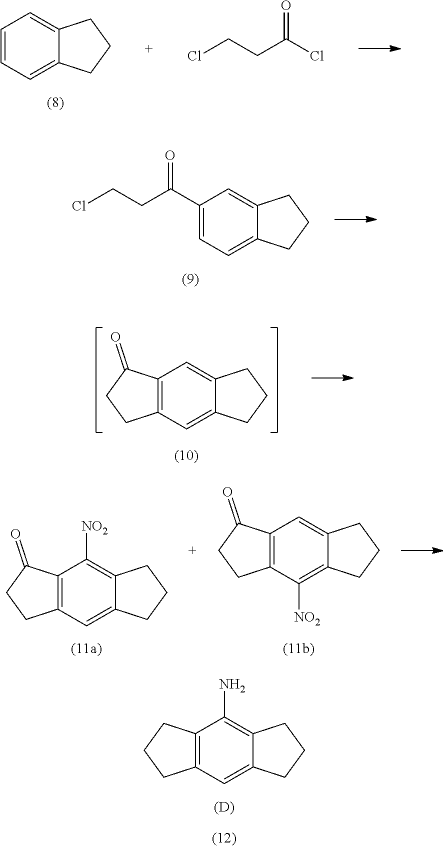

The process for the preparation of compound (D) may be as described in WO 2020/079207 A1, the contents of which are incorporated herein by reference in their entirety.

In one embodiment, the process of the fifth aspect of the invention is a process for preparing compound (A) or a salt thereof:

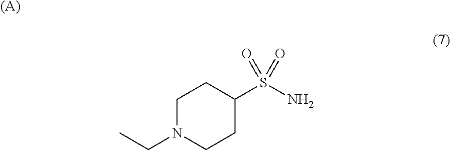

A fifth aspect of the invention provides a process for the preparation of compound (A), prepared by a process comprising the following steps:

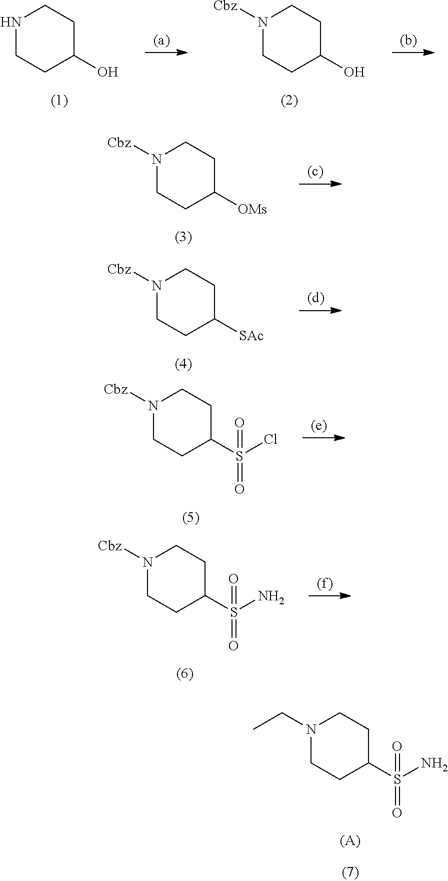

wherein Cbz is carboxybenzyl/benzyloxycarbonyl, OMs is methanesulfonate, and SAc is acetylthio.

In an exemplary embodiment of the fifth aspect of the invention, the reaction step (a) comprises contacting compound (1) with benzyl chloroformate to obtain N-carboxybenzyl-4-hydroxy piperidine compound (2):

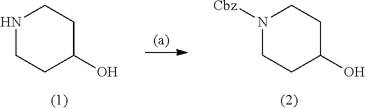 [SMILES: OC1CCNCC1 ; O=C(OCc1ccccc1)N1CCC(O)CC1]

Typically in such an embodiment, compound (1) is contacted with the benzyl chloroformate in the presence of a base and a solvent.

In an exemplary embodiment of the fifth aspect of the invention, the reaction step (b) comprises contacting compound (2) with mesyl chloride to obtain compound (3):

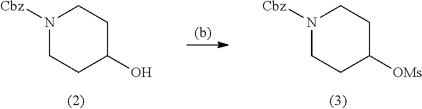 [SMILES: O=C(OCc1ccccc1)N1CCC(O)CC1 ; CS(=O)(=O)OC1CCN(C(=O)OCc2ccccc2)CC1]

Typically in such an embodiment, the compound (2) is contacted with mesyl chloride in the presence of a tertiary amine base such as triethylamine and a polar aprotic solvent such as dichloromethane.

In an exemplary embodiment of the fifth aspect of the invention, the reaction step (c) comprises contacting compound (3) with MeCOS⁻ in a solvent to obtain compound (4):

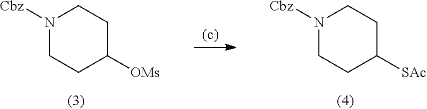

Typically in such an embodiment, the MeCOS⁻ is generated in situ by the reaction of MeCOSH with a base such as cesium carbonate. Typically in such an embodiment, the solvent is N,N-dimethylformamide.

In an exemplary embodiment of the fifth aspect of the invention, the reaction step (d) comprises contacting compound (4) with a chlorinating agent to obtain compound (5):

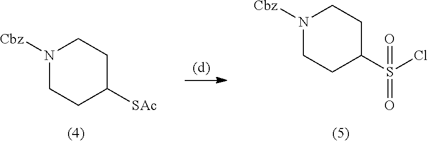

Typically in such an embodiment, the chlorinating agent is N-chlorosuccinimide.

Typically in such an embodiment, the compound (4) is contacted with the chlorinating agent in the presence of acetic acid and water.

In an exemplary embodiment of the fifth aspect of the invention, the reaction step (e) comprises contacting compound (5) with ammonia to obtain compound (6):

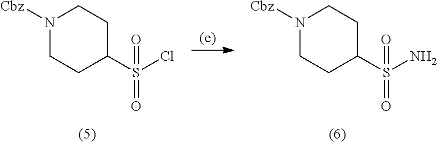 [SMILES: O=C(OCc1ccccc1)N1CCC(S(=O)(=O)Cl)CC1 ; NS(=O)(=O)C1CCN(C(=O)OCc2ccccc2)CC1]

Typically in such an embodiment, the compound (5) is contacted with ammonia in the presence of a polar aprotic solvent such as dichloromethane.

In an exemplary embodiment of the fifth aspect of the invention, the reaction step (f) comprises contacting compound (6) with acetonitrile or acetaldehyde in the presence of a catalyst and hydrogen gas, to obtain compound (A):

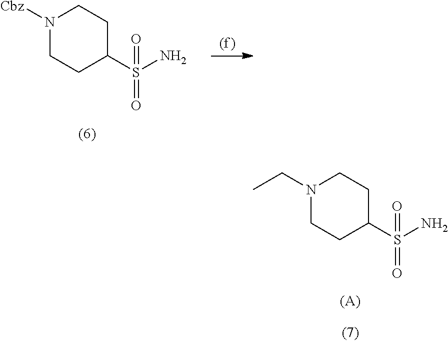 [SMILES: NS(=O)(=O)C1CCN(C(=O)OCc2ccccc2)CC1 ; CCN1CCC(S(N)(=O)=O)CC1 ; "(A)"]

Typically in such an embodiment, the compound (6) is contacted with acetonitrile in the presence of a catalyst and hydrogen gas. Typically, the catalyst is a palladium catalyst such as palladium hydroxide on carbon.

In one specific embodiment of the fifth aspect of the present invention, there is provided a process of preparing compound (A) or a salt thereof:

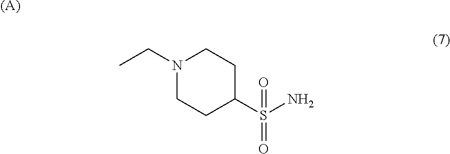

comprising the steps:
(a) converting compound (1) to compound (2):

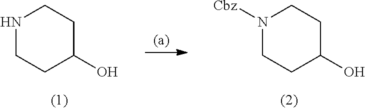 [SMILES: OC1CCNCC1 ; O=C(OCc1ccccc1)N1CCC(O)CC1]

(b) converting compound (2) to compound (3):

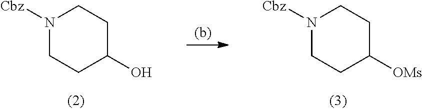 [SMILES: O=C(OCc1ccccc1)N1CCC(O)CC1 ; CS(=O)(=O)OC1CCN(C(=O)OCc2ccccc2)CC1]

(c) converting compound (3) to compound (4):

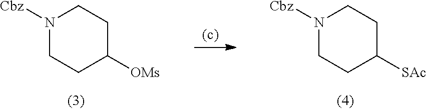

(d) converting compound (4) to compound (5):

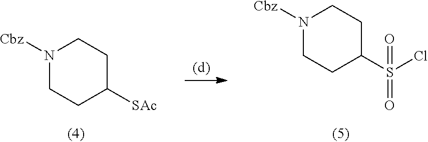

(e) converting compound (5) to compound (6)

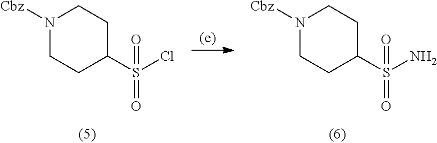 [SMILES: O=C(OCc1ccccc1)N1CCC(S(=O)(=O)Cl)CC1 ; NS(=O)(=O)C1CCN(C(=O)OCc2ccccc2)CC1]

(f) and converting compound (6) to compound (A):

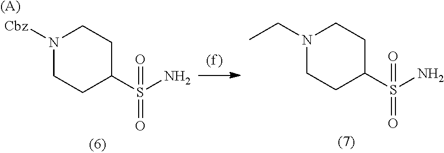

In one specific embodiment of the fifth aspect of the present invention, there is provided a process of preparing compound (A) or a salt thereof via the following steps:

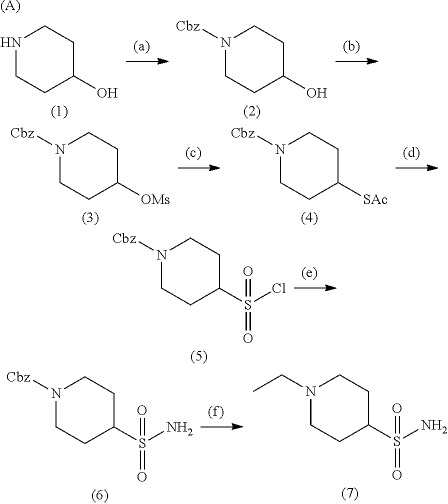

wherein Cbz is carboxybenzyl/benzyloxycarbonyl, OMs is methanesulfonate, and SAc is acetylthio.

An embodiment of the present invention provides a process to obtain compound (C) wherein compound (A) is obtained according to the fifth aspect of the invention.

The compounds used in and provided by the present invention can be used both, in their free base form and their acid addition salt form. For the purposes of this invention, a “salt” of a compound of the invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds used in and provided by the present invention can also be used both, in their free acid form and their salt form. For the purposes of this invention, a “salt” of a compound of the present invention includes one formed between a protic acid functionality (such as a carboxylic acid group or a urea group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably, any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts used in and provided by the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

The compounds, salts and solvates used in and provided by the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³J, ¹²⁴J, ¹²⁵I and ¹³¹I.

Unless stated otherwise, the compounds, salts and solvates used in and provided by the present invention may be in any polymorphic or amorphous form.

A sixth aspect of the present invention provides a pharmaceutical composition comprising the compound (C) or the salt thereof of the second aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013. Pharmaceutically acceptable excipients including adjuvants, diluents or carriers that may be used in the pharmaceutical compositions of the invention, are those conventionally employed in the field of pharmaceutical formulation.

A seventh aspect of the present invention provides compound (C) or the salt thereof of the second aspect of the invention, or the pharmaceutical composition of the sixth aspect of the invention, for use in medicine, and/or for use in the treatment or prevention of a disease, disorder or condition.

Most especially, where compound (C) is used in the treatment or prevention of a disease, disorder and condition, the compound (C) acts as an NLRP3 inhibitor.

In one embodiment, the disease, disorder or condition to be treated or prevented is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) pain; and
(xvi) any disease where an individual has been determined to carry a germline or somatic non-silent mutation in NLRP3.

Typically, the treatment or prevention of the disease, disorder or condition comprises the administration of the compound (C) or the salt thereof of the second aspect of the invention, or the pharmaceutical composition of the sixth aspect of the invention, to a subject.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder, disease or condition to be treated or prevented.

An eighth aspect of the invention provides a method of inhibiting NLRP3, the method comprising the use of the compound (C) or the salt thereof of the second aspect of the invention, or the pharmaceutical composition of the sixth aspect of the invention, to inhibit NLRP3.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

All solvents, reagents and compounds were purchased and used without further purification unless stated otherwise.
Abbreviations
Cbz: carboxybenzyl/benzyloxycarbonyl
SAc: acetylthio

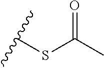 [SMILES: *SC(C)=O]

GC: gas chromatography
HPLC: high performance liquid chromatography
THF: tetrahydrofuran
MTBE: methyl tertiary butyl ether
DCM: dichloromethane
DMF: dimethylformamide
TEA: triethylamine
HDPE: high density polyethylene
NMT: No more than
Vol: volumes
AKX reagent: AQUAMICRON® AKX
% a/a: (area under peak of compound (a)/combined area under peaks of compound (a) and all other components)×100

### Experimental Methods

### NMR Methods:

NMR spectra were obtained on Bruker AV 400 MHz spectrometer (model: Advance HID) operated at room temperature (25° C.).

### GC Methods:

GC analysis was conducted on one of the following machines: Agilent 7890, 6890, or Agilent 6890N with ALS injector.

### KF Methods:

Coulometric KF (Karl Fischer) titration was run using AKX reagent on Mitsubishi CA-20 or Predicta OM1000.

### Synthesis Examples

### 1-ethyl-4-piperidinesulfonamide (7)

1-ethyl-4-piperidinesulfonamide (7) was prepared according to the reaction sequence illustrated in reaction scheme 1.

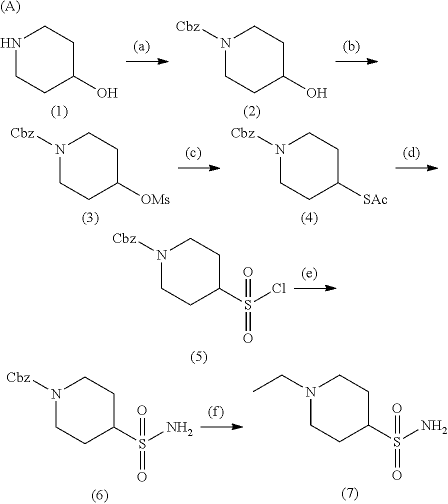

### Scheme 1. 1-ethyl-4-piperidinesulfonamide (7) Synthesis

### Reaction Scheme 1—Step (a) and (b)

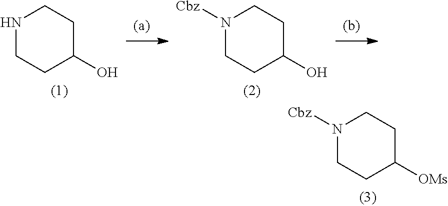

4-hydroxy piperidine (1) (46.0 Kg) was charged into the reactor at 25 to 30° C. 1,4-dioxane (226.0 L) was charged to the reactor at 25 to 30° C. The reaction mixture was stirred for 5-10 minutes and then cooled to 15 to 20° C. A 2N NaOH solution (prepared by mixing NaOH (18.4 Kg) with cold purified water (230.0 L) at 25 to 30° C. in a separate reactor) was slowly charged to the reaction mixture at 15 to 25° C. The reaction mixture was stirred for 5-10 minutes. 50% benzyl chloroformate in toluene (147.2 L) was slowly added over a period of 1-2 hours to the reaction mixture. The temperature was raised to 25 to 30° C. and stirred for 1-2 hours.

Purified water (230.0 L) was added to the reaction mixture and the reaction mixture was stirred for 10-15 min at 25 to 30° C. MTBE (230.0 L) was charged into the reactor at 30 to 35° C. The reaction mixture was stirred for 15-20 minutes at 25 to 30° C. and then allowed to settle for 20-30 minutes. The organic layer (OL-1) and aqueous layer (AL-1) were separated into different containers and AL-1 was charged back into the reactor. MTBE (230.0 L) was charged into the reactor at 25 to 30° C. The reaction mixture was stirred for 15-20 minutes at 25 to 30° C. and then allowed to settle for 20-30 minutes. The organic layer (OL-2) and aqueous layer (AL-2) were separated into different containers. OL-1 and OL-2 were combined and charged into the reactor at 25 to 30° C. Purified water (138.0 L) was charged to the reactor at 25 to 30° C. The reaction mixture was stirred for 15-20 minutes at 25 to 30° C. and then allowed to settle for 20-30 minutes. The aqueous layer (AL-3) was separated from the organic layer (OL-3).

10% NaCl solution (prepared by adding NaCl (13.80 Kg) to purified water (138.0 L) in a reactor at 25 to 30° C. with stirring) was charged to OL-3 at 25 to 30° C. The reaction mixture was stirred for 15-20 minutes at 25 to 30° C. and then allowed to settle for 20-30 minutes. The organic layer (OL-4) and aqueous layer (AL-4) were separated into different containers. OL-4 was dried with sodium sulfate (23.0 Kg). OL-4 was filtered through a Buchner funnel and washed with MTBE (46.0 L). OL-4 was distilled down to 46-92 L at 40 to 45° C. under vacuum (650 mmHg). The vacuum was released and DCM (138.0 L) was charged to the mixture and the mixture was co-distilled 35 to 40° C. under vacuum to 46-92 L. The mixture was cooled to 25 to 30° C. and the vacuum was released. DCM (552.0 L) was charged to the mixture at 25 to 30° C. and the mixture was stirred for 5-10 minutes. The reaction mixture was cooled to 20 to 25° C. TEA (127.8 L) was added at 20 to 25° C. The reaction mixture was cooled to −5 to 5° C.

Methane sulfonyl chloride (67.62 Kg) was slowly charged at −5 to 5° C. over a period of 1-2 hours. The reaction mixture was raised to 25 to 30° C. and stirred for 1-2 hours at 25 to 30° C.

The unwanted salts were filtered, washed with DCM (92.0 L) at 25 to 30° C. and sucked dry completely under vacuum at 25 to 30° C. The filtrate was charged into a reactor at 25 to 30° C. 10% sodium bicarbonate solution (prepared by adding sodium bicarbonate (23.0 Kg) to purified water (230.0 L) at 25 to 30° C.) was charged to the filtrate at 25 to 30° C. The reaction mixture was stirred for 15-20 minutes at 25 to 30° C. and then allowed to settle for 20-30 minutes. The organic layer (OL-5) and aqueous layer (AL-5) were separated into different containers and OL-5 was charged back into the reactor at 25 to 30° C.

Purified water (230.0 L) was charged into the reactor at 25 to 30° C. The reaction mixture was stirred for 15-20 minutes at 25 to 30° C. and then allowed to settle for 20-30 minutes. The organic layer (OL-6) and aqueous layer (AL-6) were separated into different containers and OL-6 was charged back into the reactor at 25 to 30° C. 10% sodium chloride solution (prepared by adding sodium chloride (11.50 Kg) to the purified water (230.0 L) at 25 to 30° C.) was charged to the reactor at 25 to 30° C. The reaction mixture was stirred for 15-20 minutes at 25 to 30° C. and then allowed to settle for 20-30 minutes.

The organic layer (OL-7) and aqueous layer (AL-7) were separated into different containers. OL-7 was dried with sodium sulfate (23.0 Kg). OL-7 was filtered through a Buchner funnel and washed with DCM (46.0 L). OL-7 was distilled down to 46-92 L at 40 to 45° C. under vacuum (650 mmHg). The vacuum was released and ethyl acetate (92.0 L) was charged to the mixture and the mixture was co-distilled 40 to 45° C. under vacuum to 46-92 L. The mixture was cooled to 30 to 40° C. and the vacuum was released. Ethyl acetate (115.0 L) was charged to the mixture at 30 to 40° C. and the mixture was stirred for 10-15 minutes at 30 to 35° C. Hexane (1150.0 L) was slowly charged to the mixture at 30 to 35° C. and the mixture was stirred for 2-3 hours at 25 to 30° C. The solid was filtered on a nutsche filter under vacuum, washed with hexane (92.0 L) at 25 to 30° C. and sucked dry completely under vacuum at 25 to 30° C. The solid material was dried in a vacuum oven at 30 to 35° C. for 6-8 hours, delumping the material every 3-4 hours.

Final Product: benzyl 4-((methylsulfonyl)oxy)piperidine-1-carboxylate

Off white colour (solid)

Output: 121.87 Kg

Yield: 85.5%

HPLC purity: 94.7%

¹H NMR: (CDCl₃400 MHz): δ 1.82-1.86 (m, 2H), δ 1.96-1.97 (m, 2H), δ 3.03 (s, 3H), δ 3.41-3.45 (m, 2H) δ 3.72-3.78 (m, 2H), δ 4.88-4.92 (m, 1H) δ 5.13 (s, 2H), δ 7.26-7.37 (m, 5H)

### Reaction Scheme 1—Step (c, d, e)

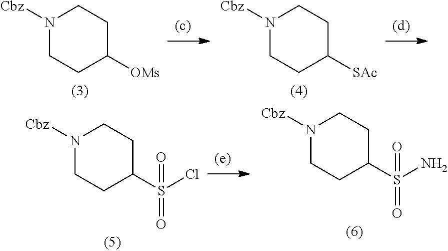

DMF was charged in to a clean and dry four neck reactor (equipped with a mechanical stirrer, nitrogen inlet, thermo pocket and reflux condenser) under nitrogen atmosphere and heated to reflux at 60 to 65° C. for 20-30 min. The temperature was reduced to 25 to 30° C., the refluxed DMF was unloaded and the reactor was dried under nitrogen and vacuum.

Benzyl 4-((methylsulfonyl)oxy)piperidine-1-carboxylate (3) (29.0 Kg) was charged to the reactor at 25 to 30° C. DMF (145.0 L) was charged to the reactor at 25 to 30° C. The reaction mixture was stirred for 5-10 minutes, cooled to 15 to 20° C. and then allowed to settle for 20-30 minutes.

Cesium carbonate 44.95 Kg was charged to the reactor at 15 to 25° C. The reaction mixture was stirred for 5-10 minutes. Thioacetic acid 10.56 Kg was charged at 15 to 25° C. The reaction mixture was raised to 45 to 50° C. and stirred for 24 hours.

The reaction mixture was cooled to 25 to 30° C. The unwanted salts were filtered through a Buchner funnel under vacuum at 25 to 30° C., washed with ethyl acetate (145.0 L) and sucked dry completely under vacuum at 25 to 30° C. The filtrate was charged back to the reactor at 25 to 30° C. and cooled to 15 to 20° C. Purified water (145.0 L) was charged to the reactor at 15-25° C. and the reaction mixture was stirred for 5-10 minutes. Ethyl acetate (145.0 L) was charged to the reactor at 25 to 30° C. The reaction mixture was stirred for 15-20 minutes at 25 to 30° C. and allowed to settle for 20-30 minutes.

The organic layer (OL-1) and aqueous layer (AL-1) were separated into different containers. AL-1 was charged into the reactor at 25 to 30° C. Ethyl acetate (145.0 L) was charged at 25 to 30° C. The reaction mixture was stirred for 15-20 minutes at 25 to 30° C. and allowed to settle for 20-30 minutes.

The organic layer (OL-2) and aqueous layer (AL-2) were separated into different containers. OL-1 and OL-2 were combined and charged into the reactor at 25 to 30° C.

A 10% NaHCO₃ solution (prepared by adding sodium bicarbonate (14.50 Kg) to purified water (145.0 L) at 25 to 30° C. and stirring well to mix) was charged to the reactor at 25 to 30° C. The reaction mixture was stirred for 15-20 minutes at 25 to 30° C. and allowed to settle for 20-30 minutes.

The organic layer (OL-3) and aqueous layer (AL-3) were separated into different containers. OL-3 was charged into the reactor at 25 to 30° C. 10% NaCl solution (prepared by adding NaCl (14.50 Kg) to purified water (145 L) at 25 to 30° C. and stirring well to mix) was charged to the reactor at 25 to 30° C. The reaction mixture was stirred for 15-20 minutes at 25 to 30° C. and allowed to settle for 20-30 minutes.

The organic layer (OL-4) and aqueous layer (AL-4) were separated into different containers. OL-4 was dried with sodium sulfate (14.50 Kg), filtered through a Buchner funnel and washed with ethyl acetate (29.0 L). The filtrate was distilled completely in the reactor until no drops at 45 to 50° C. under vacuum (650 mmHg). The vacuum was released and the mixture was cooled to 25 to 30° C.

Acetic acid (377.0 L) was charged at 25 to 30° C. to the reactor. The reaction mixture was stirred for 5-10 minutes at 25 to 30° C. Purified water (37.7 L) was charged at 25 to 30° C. The reaction mixture was stirred for 5-10 minutes at 25 to 30° C. and then cooled to 17 to 25° C. N-chlorosuccinimide (33.64 Kg) was slowly added portion wise for 1-2 hours at 18 to 25° C. The reaction mixture was stirred for 1 hour at 25 to 30° C.

The reaction mixture was cooled to 15 to 20° C. Purified water (377.0 L) was added to the reaction mixture at 15 to 20° C. and the reaction mixture was stirred for 5-10 minutes at 25 to 30° C. DCM (145.0 L) was charged to the reactor at 25 to 30° C. The reaction mixture was stirred for 10-15 minutes at 25 to 30° C. and allowed to settle for 20-30 minutes. The organic layer (OL-5) and aqueous layer (AL-5) were separated into different containers. AL-5 was charged to the reactor. DCM (145.0 L) was charged to the reactor at 25 to 30° C. The reaction mixture was stirred for 10-15 minutes at 25 to 30° C. and allowed to settle for 20-30 minutes.

The organic layer (OL-6) and aqueous layer (AL-6) were separated into different containers. OL-5 and OL-6 were combined and charged into the reactor at 25 to 30° C. Purified water (145.0 L) was charged to the reactor at 25 to 30° C. The reaction mixture was stirred for 5-10 minutes at 25 to 30° C. and allowed to settle for 25-30 minutes.

The organic layer (OL-7) and aqueous layer (AL-7) were separated into different containers. OL-7 was charged to the reactor. Part one of a 2% sodium bicarbonate solution (prepared by adding sodium bicarbonate (8.70 Kg) with purified water (435.0 L) and dividing into three equal volume parts) was charged to the reactor at 25 to 30° C. The reaction mixture was stirred for 5-10 minutes at 25 to 30° C. and allowed to settle for 25-30 minutes.

The organic layer (OL-8) and aqueous layer (AL-8) were separated into different containers. OL-8 was charged to the reactor. Part two of the above 2% sodium bicarbonate solution was charged to the reactor at 25 to 30° C. The reaction mixture was stirred for 5-10 minutes at 25 to 30° C. and allowed to settle for 25-30 minutes.

The organic layer (OL-9) and aqueous layer (AL-9) were separated into different containers. OL-9 was charged to the reactor. Part three of the above 2% sodium bicarbonate solution was charged to the RBF at 25 to 30° C. The reaction mixture was stirred for 5-10 minutes at 25 to 30° C. and allowed to settle for 25-30 minutes.

The organic layer (OL-10) and aqueous layer (AL-10) were separated into different containers. OL-10 was dried with sodium sulfate (14.50 Kg), filtered at 25 to 30° C., and washed with DCM (29.0 L). The filtrate was charged to RBF at 25 to 30° C.

The reaction mixture was cooled to −40 to −30° C. and purged with ammonia gas for 2-3 hours. The temperature was raised to 25 to 30° C. and stirred for 10-12 hours at 25 to 30° C.

The unwanted salts were filtered under vacuum at 25 to 30° C., washed with DCM (14.50 L) and sucked dry completely. The filtrate was charged into a clean and dried reactor at 25 to 30° C. and dried with sodium sulfate (14.50 Kg). The mixture was filtered at 25 to 30° C. and the sodium sulfate was washed with DCM (14.50 L). The mixture was charged through a 0.2 micron filter cartridge into a clean and dried reactor and distilled under vacuum at 35 to 40° C. down to 29-58 L.

The vacuum was released and the reaction mixture was cooled to 25 to 30° C. Ethyl acetate (58.0 L) was charged to the reactor at 25 to 30° C. and the mixture was distilled under vacuum at 35 to 40° C. down to 29-58 L. The vacuum was released and the reaction mixture was cooled to 25 to 30° C. Ethyl acetate (72.5 L) was charged to the reactor at 25 to 30° C. and the mixture was stirred for 30 min at 25 to 30° C. Hexane (36.25 L) was charged to the reactor at 25 to 30° C. and the mixture was stirred for 1-2 hours at 25 to 30° C. The solid was filtered under vacuum at 25 to 30° C., washed with hexane (58.0 L) and sucked dry completely.

Output: 11.0 Kg

Yield: 39.85%

HPLC purity: 90.5%

### Purification

Wet material (6) (53.95 Kg) was charged into a clean and dry reactor at 25 to 30° C. DCM (580 L) was charged at 25 to 30° C. and the mixture was stirred for 5-10 minutes at 25 to 30° C. Methanol (25.0 L) was charged at 25 to 30° C. and the mixture was stirred for 5-10 minutes at 25 to 30° C. Neutral alumina (174.0 Kg) was charged at 25 to 30° C. and the mixture was stirred for 1 hour at 25 to 30° C. The neutral alumina was filtered at 25 to 30° C. The salts were washed with DCM (150.0 L). The filtrate was charged in to a clean and dried reactor at 25 to 30° C. Hexane (1050 L) was charged at 25 to 30° C. and the mixture was stirred for 1-2 hours at 25 to 30° C. The precipitate was filtered under vacuum at 25 to 30° C., washed with hexane (116.0 L) and sucked dry completely. The wet material was dried under vacuum at 30 to 35° C. for 6-8 hours with delumping every 3 hours).

Final Product: 1-(benzyloxycarbonyl)-4-piperidinesulfonamide

White colour (solid powder)

Output: 41.60 Kg

Yield: 41.80%

HPLC purity: 96.10%

¹H NMR: (DMSO 400 MHz): δ 1.41-1.51 (m, 2H), δ 1.99-2.01 (m, 2H), δ 2.50-286 (m, 2H), δ 3.022-3.05 (m, 1H) δ 4.08-4.11 (m, 2H), δ 5.75 (s, 2H) δ 6.78 (s, 2H), δ 7.40-7.30 (m, 5H)

### Reaction Scheme 1—Step (f)

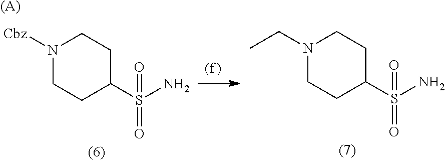

1-(benzyloxycarbonyl)-4-piperidinesulfonamide (6) (21.85 Kg) was charged to a vessel which was then purged with nitrogen. Acetonitrile (free of propionitrile) (109.8 Kg) and purified water (65.0 L) were charged to the vessel and the temperature was adjusted to 15 to 25° C. The vessel was vacuum/nitrogen purged three times at 15 to 25° C. and then charged with palladium hydroxide on carbon (20 wt %; 50% water) (0.455 Kg). The vessel was vacuum/nitrogen purged three times at 15 to 25° C. The vessel was vacuum/hydrogen purged three times at 15 to 25° C. and maintained under an atmosphere of hydrogen (ca.1 bar absolute). The reaction mixture was stirred until complete.

The vessel was vacuum/nitrogen purged three times at 15 to 25° C. and then charged with palladium hydroxide on carbon (20 wt %; 50% water) (2.265 Kg) at 15 to 25° C. The vessel was vacuum/nitrogen purged three times at 15 to 25° C. The vessel was vacuum/hydrogen purged three times at 15 to 25° C. and maintained under an atmosphere of hydrogen (ca.1 bar absolute). The reaction mixture was stirred at 15 to 25° C. until complete.

The reaction mixture was stirred at 15 to 25° C. until complete.

Once the reaction was deemed complete by GC, the vessel was purged with nitrogen and the reaction mixture was filtered through a 1 μm filter at 15 to 25° C. to remove the catalyst. The filter cake was twice washed with pre-mixed purified water and acetonitrile at 15 to 25° C.

The filtrate was charged with decolourising charcoal (activated) (4.40 Kg) and stirred at 15 to 25° C. for at least 60 minutes (target 60 to 120 minutes). The mixture was filtered through a 1 μm filter at 15 to 25° C. to remove the charcoal. The filter cake was washed twice with pre-mixed purified water and acetonitrile at 15 to 25° C. The filtrate was charged with SiliaMetS Thiol 40-63 μm 60 Å (4.515 Kg) and stirred at 15 to 25° C. for at least 60 minutes (target 60 to 120 minutes). The mixture was filtered through a 0.6 μm filter at 15 to 25° C. to remove SiliaMetS Thiol. The filter cake was twice washed with pre-mixed purified water and acetonitrile at 15 to 25° C.

The filtrate was charged to a vessel and adjusted to 50 to 6.0° C., concentrated under reduced pressure at 50 to 60° C. to ca 110 L. n-Butanol (89.8 Kg) was charged at 50 to 60° C. and the mixture was concentrated under reduced pressure at 50 to 60° C. to ca 110 L. n-Butanol (86.9 Kg) was charged at 50 to 60° C. and the mixture was concentrated under reduced pressure at 50 to 60° C. to ca 110 L. n-Butanol (88.4 Kg) was charged at 50 to 60° C. and the mixture was concentrated under reduced pressure at 50 to 60° C. to ca 90 L.

The temperature was adjusted to 15 to 25° C. and ethyl acetate (98.6 Kg) was charged at 15 to 25° C. The reaction mixture was cooled to −2 to +2° C. over at least 60 minutes (target 60 to 120 minutes). The mixture was stirred at −2 to 2° C. for at least 4 hours (target 4 to 6 hours). The solid was filtered on 20 μm filter cloth at −2 to 2° C. and washed twice with ethyl acetate, (38.1 Kg and 39.9 Kg) at −2 to 2° C.

The solid was dried at up to 60° C. under a flow of nitrogen until the n-butanol content was ≤0.5% w/w and ethyl acetate content was ≤0.5% w/w (measured by ¹H NMR spectroscopy). The dried weight of the solid 1-ethyl-4-piperidinesulfonamide (7) was measured and assayed using ¹H NMR spectroscopy.

Final Product: 1-ethyl-4-piperidinesulfonamide

Output: 12.00 Kg

Yield: 85%

GC purity: 99.7%

NMR purity: 98.7%

¹H NMR: (DMSO) 0.95 (t), 1.55 (dq), 1.80 (app t), 1.95 (app d), 2.30 (q), 2.75 (m), 2.90 (app d)

### 1,2,3,5,6,7-hexahydro-s-indacen-4-amine (12)

1,2,3,5,6,7-hexahydro-s-indacen-4-amine (12) was prepared according to the reaction sequence illustrated in Reaction Scheme 2.

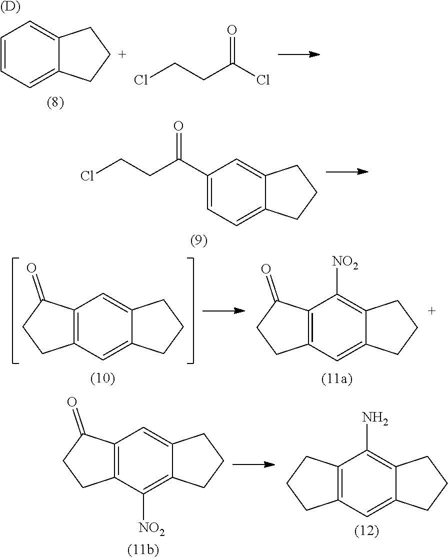

### Scheme 2. Synthesis of 1,2,3,5,6,7-hexahydro-s-indacen-4-amine (12)

### Reaction Scheme 2—Step (a)

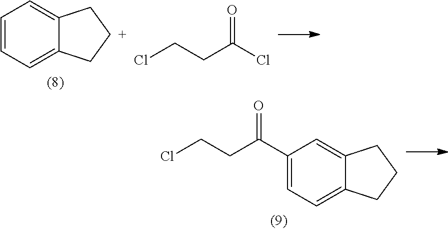

DCM (385 L) and AlCl₃ (99.86 Kg) were charged at 25 to 30° C. under a nitrogen atmosphere into a reactor clean and dry glass-lined reactor. The reaction mixture was cooled to −10° C.

3-chloropropanoyl chloride (90.99 Kg) was added slowly at −10 to −5° C. under a nitrogen atmosphere. The reaction mixture was maintained for 30 minutes at 10° C. under a nitrogen atmosphere. 2,3-dihydro-1H-indene (8) (77.00 Kg was then added slowly to the reaction mixture at −10 to −5° C. under nitrogen atmosphere.

The reaction mixture was maintained for 2 hours at 10 to 15° C.

After completion of the reaction, the reaction mixture was added slowly to a 6 N hydrochloric acid solution (prepared from water (308 L) and conc. hydrochloric acid (308 L)) at 0 to 10° C. DCM (231 L) was added and the reaction mixture temperature was raised to 30 to 35° C. The reaction mixture was stirred at 30 to 35° C. for 30 minutes and allowed to settle at 30 to 35° C. for 30 minutes. The layers were separated and the organic layer (OL-1) was kept aside. DCM (231 L) was charged to the aqueous layer at 25 to 30° C. The reaction mixture was stirred at 25 to 30° C. for 30 minutes and allowed to settle at 25 to 30° C. for 30 minutes. The layers were separated (aqueous layer (AL-1) and organic layer (OL-2)) and AL-1 was kept aside. OL-1 and OL-2 were combined at 25 to 30° C. Demineralised water (385 L) was added to the combined organic layers. The reaction mixture was stirred at 25 to 30° C. for 30 minutes and allowed to settle at 25 to 30° C. for 30 minutes. The layers were separated (aqueous layer (AL-2) and organic layer (OL-3)) and AL-2 was kept aside.

10% Saturated sodium bicarbonate solution (prepared from demineralised water (385 L) and sodium bicarbonate (38.5 Kg)) was charged to OL-3 at 25 to 30° C. The reaction mixture was stirred at 25 to 30° C. for 30 minutes and allowed to settle at 25 to 30° C. for 30 minutes. The layers were separated (aqueous layer (AL-3) and organic layer (OL-4)) and AL-3 was kept aside. OL-4 was dried over anhydrous Na₂SO₄ (38.5 Kg) and the anhydrous Na₂SO₄ was washed with DCM (150 L) at 25 to 30° C.

The solvent was distilled under vacuum at below 35 to 40° C. until 5% remained. n-hexane (308 L) was charged to the reaction mixture at 35 to 40° C. and the solvent was distilled completely at 35 to 40° C. until no condensate drops were formed. N-hexane (150 L) was charged to the reaction mixture at 35 to 40° C. and the reaction mixture was cooled to 5 to 10° C. and maintained at 5 to 10° C. for 30 minutes.

The solid product was filtered, washed with cooled hexane (77 L), and dried in a hot air oven at 40 to 45° C. for 6 hours to afford the product.

Final Product: 3-chloro-1-(2,3-dihydro-1H-inden-5-yl)propan-1-one (9)

Output: 120.5 Kg

Yield: 88.63%

HPLC purity: 99.3%

Moisture content: 0.09%

¹H NMR: (500 MHz, CDCl3): δ 7.81 (S, 1H), 7.76 (d, 1H), 7.31 (d, 1H), 3.93 (t, 2H), 3.45 (t, 2H), 2.97 (t, 4H), 2.15 (q, 2H)

### Reaction Scheme 2—Step (b) and Step (c)

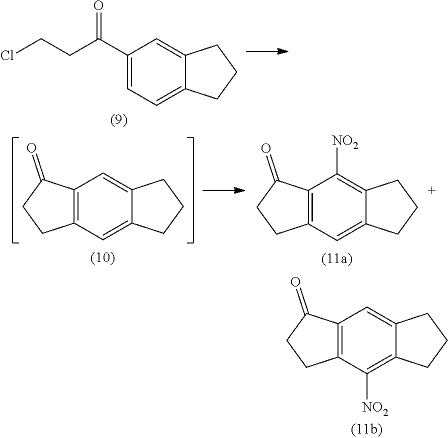

Sulfuric acid (300.0 L) was charged at 25 to 30° C. into a 2.0 KL clean and dry glass-lined reactor. 3-chloro-1-(2,3-dihydro-1H-inden-5-yl)propan-1-one (9) (60.0 Kg) was charged lot wise at 25 to 30° C. and the reaction mixture was maintained for 30 minutes at 25 to 30° C. The reaction mixture was slowly heated to 65 to 70° C. and maintained at 65 to 70° C. for 24 hours. The absence of 3-chloro-1-(2,3-dihydro-1H-inden-5-yl)propan-1-one (9) was confirmed by HPLC (Limit: ≤1.0%).

Then the reaction mixture was cooled to 0 to 5° C. A nitration mixture*¹ was added slowly at 0 to 5° C. and the reaction mixture was maintained at 0 to 5° C. for 1 hour. The reaction mixture was maintained at 0 to 5° C.

Demineralised water (900.0 L) was charged at 25 to 30° C. into a 2.0 KL clean and dry glass-lined reactor. The water was cooled to 0 to 5° C. The reaction mixture was added slowly added to the reactor at 0 to 5° C. Toluene (480.0 L) was added and the temperature was raised to 30 to 35° C. The reaction mixture was maintained at 30 to 35° C. for 30 minutes and allowed to settle at 30 to 35° C. for 30 minutes. The reaction mixture was filtered through a Celite® bed (prepared with Celite® (6.0 Kg) and toluene (30.0 L)). The Celite® bed was washed with toluene (60.0 L). The solid was filtered and sucked dry for 30 min.

The reaction mixture was charged to a 2.0 KL clean and dry glass-lined reactor. The reaction mixture was allowed to settle at 30 to 35° C. for 30 minutes. The layers were separated (aqueous layer (AL-1) and organic layer (OL-1)) and OL-1 was kept aside. Toluene (60.0 L) was charged to AL-1. The reaction mixture was stirred at 35 to 40° C. for 30 minutes and allowed to settle at 35 to 40° C. for 30 minutes. The layers were separated (aqueous layer (AL-2) and organic layer (OL-2)) and OL-2 was kept aside. OL-1 and OL-2 were combined to form OL-3.

A 5% saturated sodium bicarbonate solution (prepared from demineralised water (300.0 L) and sodium bicarbonate (15.0 Kg)) was slowly charged to OL-3 at 30 to 35° C. The reaction mixture was stirred at 35 to 40° C. for 30 minutes and allowed to settle at 35 to 40° C. for 30 minutes. The reaction mixture was filtered through a Celite® bed (prepared with Celite® (6.0 Kg) and demineralised water (60.0 L)). The Celite® bed was washed with toluene (60.0 L).

The reaction mixture was charged to a 3.0 KL clean and dry glass-lined reactor. The reaction mixture was allowed to settle at 30 to 35° C. for 30 minutes. The layers were separated (aqueous layer (AL-3) and organic layer (OL-4)) and OL-4 was kept aside.

Toluene (60.0 L) was charged to AL-3. The layers were separated (aqueous layer (AL-4) and organic layer (OL-5)) and OL-5 was kept aside. OL-4 and OL-5 were combined to form OL-6. Brine solution (prepared from demineralised water (300.0 L) and sodium chloride (12.0 Kg) at 25 to 30° C. The reaction mixture was stirred at 30 to 35° C. for 30 minutes and allowed to settle at 30 to 35° C. for 30 minutes. The layers were separated (aqueous layer (AL-5) and organic layer (OL-7)) and OL-7 was kept aside. OL-7 was dried over anhydrous Na₂SO₄ (9.0 Kg) and the anhydrous Na₂SO₄ was washed with toluene (30.0 L) at 25 to 30° C. The solvent was distilled under vacuum at below 40 to 45° C. until 5% remained. Methanol (60.0 L) was charged to the reaction mixture at 40 to 45° C. and down to 60 L of reaction mass.

Methanol (120.0 L) was charged to the reaction mixture at 40 to 45° C. and the reaction mixture was cooled to 5 to 10° C. and maintained at 5 to 10° C. for 30 minutes. The solid product was filtered, washed with cooled methanol (30.0 L), and dried in a hot air oven at 40 to 45° C. for 6 hours to afford the product.

*1: To prepare the nitration mixture, sulfuric acid (27.0 L) was charged at 25 to 30° C. into a 160 L clean and dry glass-lined reactor. The reaction mixture was cooled to 0 to 5° C. Nitric acid (27.0 L) at 0 to 5° C. was added slowly and the reaction mixture was maintained for 30 minutes at 0 to 5° C. to afford the nitration mixture.

Final Product: 8-nitro-1,2,3,5,6,7-hexahydro-s-indacen-1-one (11a) and 4-nitro-1,2,3,5,6,7-hexahydro-s-indacen-1-one (11b)

Combined Output (11a+11b): 38.87 Kg

Combined Yield (11a+11b): 62.24%

Weight ratio (11a:11b): 9:1

HPLC purity: 95.9%

Moisture content: 0.19%

¹H NMR: (500 MHz, CDCl₃): δ7.44 (S, 1H), 2.21 (m, 2H), 2.78 (t, 2H), 3.02 (m, 4H), 3.13 (t, 2H)

### Reaction Scheme 2—Step (d)

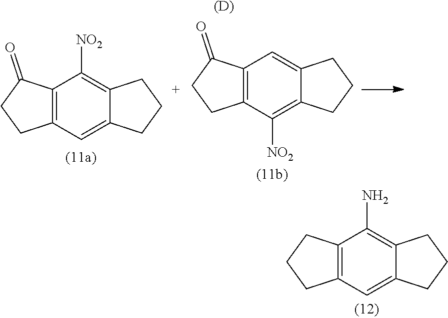

A mixture of 8-nitro-1,2,3,5,6,7-hexahydro-s-indacen-1-one (11a) and 4-nitro-1,2,3,5,6,7-hexahydro-s-indacen-1-one (11b) (9:1 ratio; 27.0 Kg) at 25 to 30° C. was charged into a 600 L clean and dry pressure reactor.

Methanol (270 L) was charged at 25 to 30° C. Methane sulfonic acid (14.3 Kg) was slowly charged at 25 to 30° C. and the reaction mixture was maintained for 30 minutes. 15% Pd(OH)₂ slurry (60% wet)*² was added.

The reaction mixture was degassed under vacuum and filled with an argon atmosphere (0.5 Kg) three times. The reaction mixture was degassed under vacuum and filled with a hydrogen atmosphere (0.5 Kg) three times. Then the reaction mixture was stirred under hydrogen pressure (100 Psi) at room temperature for 32 hours.

After completion of the reaction, the reaction mixture was cooled to 25 to 30° C. The reaction mixture was degassed under vacuum and filled with nitrogen atmosphere (0.5 Kg) three times.

The reaction mixture was filtered through a candy filter to remove Pd(OH)₂, followed by a micro filter and the bed was washed with methanol (54 L). 95% of the solvent was distilled off under vacuum at below 45 to 50° C. Demineralised water (135 L) was charged into the reaction mixture at 25 to 30° C. and maintained for 30 minutes. The reaction mixture was cooled to 5-10° C. The pH was adjusted to about 9-10 with 2 N aqueous NaOH solution (prepared from NaOH (6.48 Kg) and demineralised water (81 L)) and the reaction mixture was stirred for 30 minutes. Then toluene (135 L) was charged to the reaction mixture and the reaction mixture was stirred for 30 minutes. The reaction mixture was stirred for a further 30 minutes, whilst bringing the temperature up to 25 to 30° C. The reaction mixture was allowed to settle for 30 minutes, whilst the temperature was maintained at 25 to 30° C.

The reaction mixture was filtered through a Celite® bed (prepared with Celite® (5.4 Kg) and toluene (13.5 L). The Celite® bed was washed with toluene (54 L).

The layers were separated (aqueous layer (AL-1) and organic layer (OL-1)) and OL-1 was kept aside. Toluene (54 L) was added to AL-1 at 25 to 30° C. The reaction mixture was stirred at 25 to 30° C. for 30 minutes and allowed to settle at 25 to 30° C. for 30 minutes. The layers were separated (aqueous layer (AL-2) and organic layer (OL-2)) and AL-2 was kept aside. Toluene (54 L) was added to AL-1 at 25 to 30° C. A brine solution (prepared with demineralised water (135 L) and sodium chloride (54 Kg)) was charged to the combined organic layers (OL-1 and OL-2) at 25 to 30° C. The reaction mixture was stirred at 25 to 30° C. for 30 minutes and allowed to settle at 25 to 30° C. for 30 minutes.

The layers were separated (aqueous layer (AL-3) and organic layer (OL-3)) and AL-3 was kept aside. Charcoal (1.3 Kg) was added to OL-3 and the temperature was raised to 35-40° C. and maintained at 35 to 40° C. for 30 minutes. The reaction mixture was filtered through a Celite® bed (prepared with Celite® (5.4 Kg) and toluene (54 L)) at 35 to 40° C. The Celite® bed was washed with toluene (54 L). The organic layer was dried over anhydrous Na₂SO₄ (13.5 Kg). The Na₂SO₄ was washed with toluene (27 L).

The solvent was distilled under vacuum at below 35 to 40° C. until 5% remained. Methanol (40.5 L) was charged to the reaction mixture at 35 to 40° C. and distilled until 5% remained. Methanol (97.2 L) and water (10.8 L) were charged to the reaction mixture at 35 to 40° C. The reaction mixture was heated to 50 to 55° C., stirred for 1 hour at 50 to 55° C., slowly cooled to 0 to 5° C. and maintained at 0 to 5° C. for 30 minutes.

The solid product was filtered and washed with cold methanol (13.5 L), and dried in a hot air oven at 40 to 45° C. for 6 hours to afford the product.

*2: To prepare the 15% Pd(OH)₂ slurry, 20% Pd(OH)₂ on carbon (60% wet; 4.05 Kg) was added to methanol (27 L).

Final product: 1,2,3,5,6,7-hexahydro-s-indacen-4-amine (12)

Output: 11.3 Kg

Yield: 41.85%

HPLC purity: 98.1%

Moisture content: 0.10

¹H NMR: (400 MHz, DMSO-d₆): δ 6.38 (S, 1H), 4.45 (S, 2H), 2.75 (t, 4H), 2.58 (t, 4H), 1.98 (t, 4H).

Purification (A) of 1,2,3,5,6,7-hexahydro-s-indacen-4-amine (12)

1,2,3,5,6,7-Hexahydro-s-indacen-4-amine (12) (54.5 Kg) was charged at 25 to 30° C. into a 250 L clean and dry reactor. Toluene (27.2 L) was charged at 25 to 30° C. and the reaction mixture was stirred at 25 to 30° C. for 30 minutes. Methanol (163 L) was charged to the reaction mixture at 25 to 30° C. The reaction mixture was stirred at 25 to 30° C. for 30 minutes, cooled to −5 to 0° C., and stirred at −5 to 0° C. for 30 minutes. The solid product was filtered, washed with cold methanol (54.5 L), and dried at 40 to 45° C. for 6 hours.

Final Product: 1,2,3,5,6,7-hexahydro-s-indacen-4-amine (12)

Output: 40.5 Kg

Yield: 74.31%

HPLC purity: 99.5%

Moisture content: 0.3%

¹H NMR: (400 MHz, DMSO-d₆): δ 6.33 (s, 1H), 4.53 (s, 2H), 2.72 (t, 4H), 2.57 (t, 4H), 1.98 (t, 4H).

### 1-Ethyl-N-((1,2,3,5,6,7-hexahydro-s-indacen-4-yl)carbamoyl)piperidine-4-sulfonamide (Potassium Salt) (14)

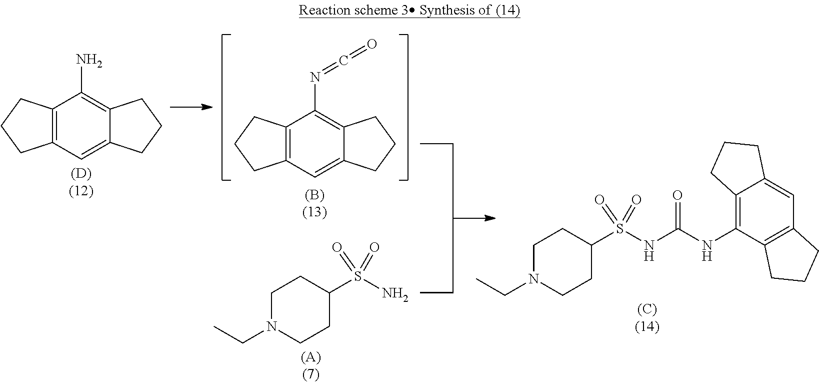

1,2,3,5,6,7-hexahydro-s-indacen-4-amine-isocyanate (13) Preparation in a Batch Mode:

1,2,3,5,6,7-hexahydro-s-indacen-4-amine (12) (1.00 g, 1.00 equiv) was dissolved in toluene (9.60 g) in a 50 mL reactor at 10-20° C. N,N-diisopropylethylamine (2.25 g, 3.00 equiv) was added followed by the 20 wt % phosgene solution (4.28 g, 1.50 equiv) over 3 minutes and the formed suspension was further stirred for 30 minutes at 10-20° C. The reaction mixture was washed with saturated NaHCO₃ solution (5.0 mL) and water (5.0 mL). The layers were separated to give 1,2,3,5,6,7-hexahydro-s-indacen-4-amine-isocyanate in toluene (OL-1, ca. 20 mL, contains 1,2,3,5,6,7-hexahydro-s-indacen-4-amine (12) (5.77 mmol). The obtained solution OL-1 is used in the next step (Coupling of indacenamine-isocyanate (12) with 1-ethyl-4-piperidinesulfonamide (7)) to yield (14) in ca. 80% overall yield.

1,2,3,5,6,7-hexahydro-s-indacen-4-amine-isocyanate (13) Preparation in a Flow Mode:

### Feed Solutions Preparation:

Feed solution A: 1,2,3,5,6,7-hexahydro-s-indacen-4-amine (12) (43.31 g) was dissolved in toluene (206.69 g) to give a 0.90 M solution.

Feed solution B: Potassium carbonate (103.5 g) was dissolved in water (950 g) to give a 0.75 M solution.

Feed A (0.70 mL/min, 1.10 equiv), 20% w/w phosgene solution toluene (0.45 mL/min, 1.50 equiv) and Feed B solution (2.35 mL/min, 3.10 equiv) was dosed simultaneously at 0 to 10° C. (Internal temperature) in a reactor 1 (ca. 25 mL). Residence time in reactor 1 is 5-10 minutes. The biphasic solution from reactor 1 is continuously pumped out and layers are separated continuously to give organic layer (OL-1) with 1,2,3,5,6,7-hexahydro-s-indacen-4-amine isocyanate (13) and aqueous layer (AL-1) that is directed to the waste. Organic layer OL-1 is collected over 81 minutes at steady state to afford ca. 90 mL of 1,2,3,5,6,7-hexahydro-s-indacen-4-amine (12) (51 mmol). The obtained solution OL-1 is used in the next step.

Coupling of Indacenamine-Isocyanate (12) with 1-ethyl-4-piperidinesulfonamide (7):

1-ethyl-4-piperidinesulfonamide (7) (8.88 g, 46 mmol, 1.0 equiv) was charged to a vessel. Tetrahydrofuran (62.52 g) was charged to the vessel and the mixture was adjusted to 20 to 25° C. The mixture was stirred for at least 20 minutes at 20 to 25° C. until clumps disappeared and homogenous suspension was formed. Potassium tert-butoxide (1.05 M, 43.98 mL, 46 mmol) was charged to the vessel over 90 to 120 minutes, maintaining the temperature at 20 to 25° C. and the mixture stirred for 2 to 4 hours at 20 to 25° C. to give a thick, white suspension.

The organic layer OL-1 containing 1,2,3,5,6,7-hexahydro-s-indacen-4-amine-isocyanate (13) (51 mmol of 1,2,3,5,6,7-hexahydro-s-indacen-4-amine (12), ca. 90 mL) prepared in a batch or a flow mode was added to the formed white suspension in toluene over 2 hours by keeping 20-25° C. The reaction mixture quickly became a well stirrable suspension and at the end of addition a slightly turbid brown solution. The reaction mixture was stirred further 1-2 h at 20 to 25° C. The water content was analysed by KF and conversion of 1,2,3,5,6,7-hexahydro-s-indacen-4-amine confirmed by LC/MS or HPLC analysis (typically >95%). Optionally a clear filtration via Celite layer (G3 filter) is performed. Water (4.44 g, 0.5 V) was added to the reaction mixture at 25 to 40° C. dropwise over 2 hours. Solids started to crystallize at about 0.5-1 wt % water content. At the end of dosing a suspension was formed. The reaction mixture was cooled to 0 to 5° C. (IT) over 1 h and stirred further for 16 h at 0 to 5° C. Solids were filtered through a G3 filter and washed with toluene/THF (1/1 by volume, 44.4 mL) mixture.

The solid was dried at up to 50° C., 10-20 mbar under a flow of nitrogen over 12 h. The dried weight of the crude solid was measured, identified and analysed using ¹H NMR spectroscopy and HPLC.

Final Product: 1-ethyl-N-((1,2,3,5,6,7-hexahydro-s-indacen-4-yl)-carbamoyl)piperidine-4-sulfonamide (Potassium Salt) (14)

Output: ca. 16.0 g

Yield: ca. 80%

NMR purity: >97%

HPLC purity: >99%

Recrystallization of 1-ethyl-N-((1,2,3,5,6,7-hexahydro-s-indacen-4-yl)-carbamoyl)piperidine-4-sulfonamide (Potassium Salt) (14)

Crude 1-ethyl-N-((1,2,3,5,6,7-hexahydro-s-indacen-4-yl)carbamoyl)piperidine-4-sulfonamide (potassium salt) (14) (15.00 g) was charged to a reaction vessel. Methanol (33.55 g) was charged to the vessel followed by acetonitrile (33.55 g) and the temperature was adjusted to 15 to 25° C. as required with stirring for 10 to 20 minutes (until a homogeneous cloudy solution with no lumps of solid present was formed). The solution was filtered through a 1 μm filter at 15 to 25° C. The filter was washed with methanol/acetonitrile mixture (7-59 g) at 15 to 25° C. and further acetonitrile (64.0 g) was added followed by seed crystals (0.138 g) of (14) in acetonitrile (ca. 1 g). Suspension was formed.

The solution was concentrated to ca. 122 mL at 25 to 35° C. Acetonitrile (54.32 g) was charged to the mixture and the solution was concentrated to ca. 122 L at 25 to 35° C. Acetonitrile (52.53 g) was charged to the mixture and the mixture was concentrated to ca. 122 mL at ≤35° C. The mixture was analysed for residual methanol content. Pass criterion ≤0.3% w/w methanol. Acetonitrile (53.45 g) was charged to the vessel and the temperature was adjusted to 15 to 25° C. The slurry was aged for at least 1 hour (target 1 to 2 hours) at 15 to 25° C. and then filtered over 20 μm cloth at 15 to 25° C. The filter cake was twice washed with acetonitrile (43.39 g) at 15 to 25° C. The solid was dried at up to 50° C. under a flow of nitrogen to yield 13.75 g (92%) of the white solid.

Final Product: 1-ethyl-N-((1,2,3,5,6,7-hexahydro-s-indacen-4-yl)-carbamoyl)piperidine-4-sulfonamide (Potassium Salt) (14)

Output: 13.75 g

Yield: 92%

HPLC purity: 99.7%

1. A process of preparing compound (C) or a salt thereof, comprising the step of contacting compound (A) with compound (B) in the presence of a solvent and a base, to obtain compound (C) or a salt thereof

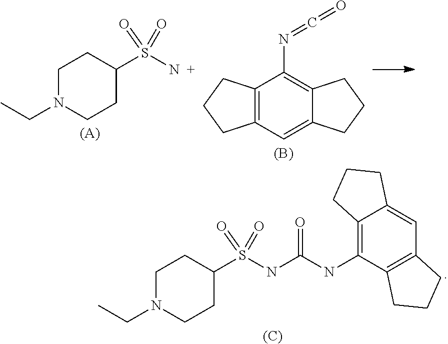

2. The process of claim 1, wherein the solvent for contacting compound (A) with compound (B) is selected from toluene, anisole, cyclopentyl methyl ether, ethylbenzene, isopropyl acetate, isobutyl acetate, 2-methyl tetrahydrofuran, water, t-butanol, ethyl acetate, methyl acetate, xylene, tetrahydrofuran dimethyl sulfoxide, acetonitrile, t-butyl methyl ether, N-methyl pyrrolidine, N-ethyl pyrrolidone, heptane, cyclohexane, acetone, or any combination thereof.

3. The process of claim 1, wherein the solvent for contacting compound (A) with compound (B) is toluene or toluene in combination with water, tert-butanol, tetrahydrofuran, dimethyl sulfoxide or acetonitrile.

4. The process claim 1, wherein the solvent for contacting compound (A) with compound (B) is toluene and tetrahydrofuran

5. The process of claim 1, wherein the base for contacting compound (A) with compound (B) is selected from potassium tert-butoxide, potassium hydroxide or any other basic potassium salt.

6. The process of claim 1, wherein the base for contacting compound (A) with compound (B) is selected from potassium tert-butoxide or potassium hydroxide.

7. The process of claim 1, wherein the base for contacting compound (A) with compound (B) is potassium tert-butoxide.

8. A process of preparing compound (B) wherein compound (D) is converted into compound (B):

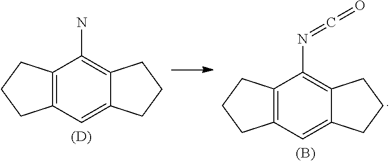

9. The process according to 8, wherein compound (D) is converted into compound B using a reaction mixture of compound (D) with phosgene, triphosgene, carbonyldiimidazole, or di-tert-butyl dicarbonate in the presence of a base and a solvent.

10. The process of claim 9, wherein
the solvent is selected from toluene, anisole, cyclopentylmethylether, ethylbenzene, isopropyl acetate, isobutyl acetate, 2-methyl tetrahydrofuran, water, ethyl acetate, methyl acetate, xylene, tetrahydrofuran or dimethyl sulfoxide, acetonitrile, t-butyl methyl ether, diethyl ether, dichloromethane, 1,2-dichloroethane, chloroform, N-methyl pyrrolidine, N-ethyl pyrrolidone, heptane, cyclohexane or any combination thereof; and
the base is a tertiary amine, such as N,N-diisopropylethylamine, triethylamine, or tributylamine or the base is an inorganic base such as potassium carbonate, potassium hydroxide, or sodium carbonate.

11. The process of claim 9, wherein
the solvent is selected from toluene or toluene in combination with water, acetonitrile, or tetrahydrofuran; and
the base is selected from N,N-diisopropylethylamine, trimethylamine, tributylamine, potassium carbonate, potassium hydroxide, or sodium carbonate.

12. The process of claim 9, wherein the solvent is toluene and/or water, and the base is N,N-diisopropylethylamine, trimethylamine or potassium carbonate.

13. The process of a claim 9, wherein the solvent is toluene and the base is N,N-diisopropylethylamine or potassium carbonate.

14. (canceled)

15. The process of a claim 1, further comprising preparing compound (B), wherein compound (D) is converted into compound (B).

16. (canceled)

17. The process of claim 8, wherein the process is performed in a continuous mode.

18. The process of claim 1, wherein compound (C) is isolated using an antisolvent.

19. The process of claim 18, wherein the antisolvent is selected from acetonitrile, any alcohol or water.

20. The process of claim 1, wherein compound (C) is isolated using a wash solvent.

21. The process of claim 20, wherein the wash solvent is selected from tetrahydrofuran, toluene, dimethylsulfoxide, or acetonitrile.

22. (canceled)

23. (canceled)

24. A process according to claim 8, further comprising preparing compound of formula (D), wherein compound (D) is prepared via the following steps:

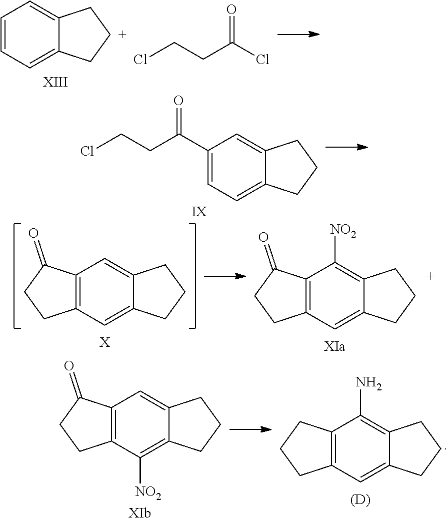

25. A process according to claim 1, further comprising preparing compound of formula (A), wherein compound (A) is prepared via the following steps: 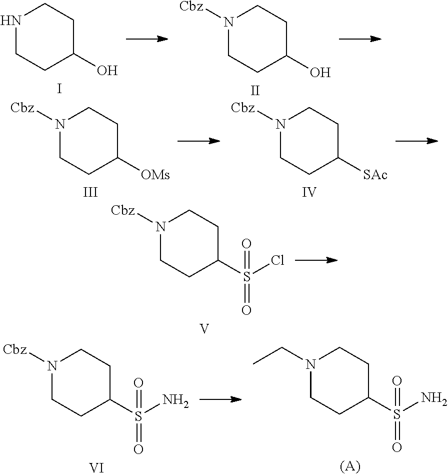
wherein Cbz is carboxybenzyl/benzyloxycarbonyl, OMs is methanesulfonate, and SAc is acetylthio.

26. (canceled)
